(19) United States
(12) Patent Application Publication
(10) Pub. No.: US 2025/0312573 A1
(43) Pub. Date: Oct. 9, 2025

(54) Holder for Holding a Guidewire

(71) Applicant: Straub Medical AG, Wangs (CH)

(72) Inventor: Tom Lavrijsen, Sargans (CH)

(21) Appl. No.: 18/866,123

(22) PCT Filed: May 24, 2022
(86) PCT No.: PCT/EP2022/064023

§ 371 Date: Nov. 15, 2024

(51) Int. Cl. A61M 25/09; A61B 17/00; A61B 17/32; A61B 17/3207

(52) U.S. Cl. CPC A61M 25/09041; A61B 17/320758; A61B 2017/00991; A61B 2017/320052

(57) ABSTRACT

A holder has a capturing portion, a connecting portion, and a translating portion. The capturing portion is connected to the translating portion. The translating portion connects to the connecting portion. The capturing portion captures the guidewire to restrict movement of the guidewire relative to the capturing portion. The connecting portion connects the holder to the handle to restrict movement between the connecting portion and the handle. The translating portion translates the capturing portion relative to the connecting portion, while the guidewire is captured by the capturing portion, wherein the translating portion is configured to shorten and extend the holder in the length direction in shortened and extended states, respectively, such that the holder is configured to facilitate retraction and advancement of the guidewire in the length direction while restricting rotation of the guidewire relative to the handle.

### CROSS REFERENCE TO RELATED APPLICATIONS

This application is a U.S. national phase of International Application No. PCT/EP2022/064023, filed May 24, 2022, which is incorporated herein by reference in its entirety.

### TECHNICAL FIELD

The present disclosure concerns a holder for holding a guidewire and positioning a catheter relative to the guidewire during a vascular treatment. The present disclosure also concerns a set comprising a handle and a holder, and an atherectomy or thrombectomy system.

### BACKGROUND

In the prior art, a so-called guidewire torque device, which is basically a grip which helps to grip the guidewire external to the body and which is usually held behind the handle is known. For example, WO 2018/090992 A1 discloses such guidewire torque control device.

During vascular treatments, an occlusion or other lesion is to be treated along its length. As such, translation of the catheter at the distal tip of which a “treating” entity is provided is needed during the procedure, usually realized by way of a back and forth movement of the catheter. As a guidewire is used and the guidewire is anchored in the vasculature and is, thus, stationary relative the patient's body, relative movement between the guidewire and the remaining apparatus, in particular the catheter, is required. Usually, an additional assistant is needed for gripping the guidewire and for allowing for relative back and forth movement between the catheter and the guidewire. At the same time, the assistant would ensure that any rotation of the guidewire, as such resulting from rotation of an entity within the catheter, is restricted.

### SUMMARY OF THE DISCLOSURE

The present disclosure may be regarded as providing a simplified and, at the same time, more accurate and comfortable system for facilitating back and forth movement of a catheter for treating an occlusion during a procedure.

To address this, claim 1 is directed to a holder, the holder having a capturing portion, a connecting portion and a translating portion, wherein the capturing portion is connected to the translating portion, and the translating portion is connected to the connecting portion. The capturing portion is configured for capturing the guidewire to restrict movement, in particular rotation and translation, of the guidewire relative to the capturing portion. The connecting portion is configured for connecting the holder to the handle to restrict movement, in particular rotation, between the connecting portion and the handle. The translating portion is configured for translating the capturing portion relative to the connecting portion between a shortened state and an extended state of the holder along a length direction, while the guidewire is captured by the capturing portion, wherein the translating portion is configured to shorten and extend the holder in the length direction in the retracted and the advanced states, respectively, of a catheter (relative to the guidewire), while rotation of the translating portion relative to the capturing portion and the connecting portion is restricted, such that the holder is configured to facilitate relative movement between the catheter and the guidewire, while restricting rotation of the guidewire relative to the holder/handle.

Accordingly, the connecting portion connects to the handle; and the capturing portion, which may be regarded as a grip for the guidewire, is connected to the guidewire, wherein the capturing portion can be translated relative to the connecting portion via the translating portion. Hence, by translating the connecting portion relative to the capturing portion, the catheter can be translated, together with the handle, relative to guidewire, while the guidewire is “held” by the holder. Consequently, it may no longer be necessary for an assistant or additional physician to hold the guidewire. This may make vascular treatments more precise, convenient and simple.

The holder of the present disclosure can be regarded as a guidewire torque device.

Optionally, the translating portion is configured to house a section of the guidewire when the translating portion is in the shortened state and/or the extended state. By housing such proximal portion of the guidewire in the translating portion, smooth translation of the guidewire and reduced mechanical hurdles can be supported. For example, housing a section of the guidewire in the translating portion can avoid that the guidewire is fastened with knots or subjected to any entanglement.

Optionally, the translating portion includes at least two telescopic elements, which are configured to telescope relative to each other in the length direction, the capturing portion remaining stationary relative to one of the at least two telescopic elements and/or the connecting portion remaining stationary relative to the other one of the at least two telescopic elements. Put differently, the capturing portion together with one of the telescopic elements, while the other telescopic element does not move relative to the connecting portion and the handle. By providing a telescopic configuration, guided movement of the translating portion can be provided. Telescopic elements provide a practical implementation of translating portions.

Optionally, the telescopic elements may comprise tubes. In other words, a first telescopic tube may telescope relative to a second telescopic tube. The telescopic elements, however, do not need to have a circular cross-section and do not need to be tubes. Other cross-sections are possible. Telescopic elements may comprise rails or slides.

Optionally, each of the telescopic elements has a length between 50 mm and 300 mm. As a typical lesion may have a length up to 200 mm, it is advisable to allow at least for back-and-forth movement in this range. Ideally, the telescopic elements each have a length of at least 100 mm. This may allow for full coverage of an occlusion during a treatment.

Optionally, the telescopic elements are tubular and each have a diameter of less than 20 mm, optionally less than 15 mm. By way of such dimensions, the guidewire can be housed appropriately within the telescopic elements, while stable translating portion is provided.

Optionally, the at least two telescopic elements comprise an inner telescopic element and an outer telescopic element at least partially receiving the inner telescopic element. Further optionally, the outer telescopic element has an inner diameter between 8 mm and 13 mm, and the inner telescopic element has an outer diameter between 5 mm and 10 mm, if tubes are used. Alternatively or additionally, an inner cross-sectional area of the outer telescopic element may be between 50 and 140 mm², while an outer cross-sectional area of the inner telescopic element may be between 20 and 80 mm², if the entire area inside the telescopic element is considered as cross-sectional area, in a cross-sectional view. Accordingly, a stable and at the same time minimised configuration can be obtained.

Optionally, one of the at least two telescopic elements at least partially comprises a receiving feature, optionally a channel, extending along a length direction of the telescopic elements and the other one of the at least two telescopic elements comprising an engagement feature, optionally a protrusion, at least partially received in the receiving feature, such that rotation of the telescopic elements relative to each other is restricted, but translation of the telescopic elements relative to each other in the length direction is possible. Accordingly, by way of the receiving feature extending in the length direction at one of the telescopic elements and the corresponding engagement feature at the other telescopic element, it can be ensured that the telescopic elements are movable relative to each other only in the length direction, i.e. can translate relative to each other, without rotation of the telescopic elements relative to each other. This restricts rotation of the guidewire, as received in the capturing portion, relative to the connecting portion, i.e. relative to the handle. Consequently, by providing the translating portion according to this embodiment, the translating portion may be configured for translating the connecting portion relative to the capturing portion in the length direction in an efficient and simple way.

The connecting portion may be configured to establish a friction fit relative to the handle. Accordingly, (this part of) the holder can be firmly fixed to the handle, as the connecting portion does not rotate relative to the handle.

Additionally or alternatively, the capturing portion is configured to establish a friction fit relative to the guidewire. By way of the friction fit between the capturing portion and the guidewire, the guidewire is fixed relative to the capturing portion in the length direction as well as in the circumferential direction.

The capturing portion may be configured to establish a form fit relative to the translating portion. In other words, the capturing and translating portions may be, in the holder, in a form fit relative to each other. Accordingly, no rotation between the capturing portion and the translating portion may be possible.

Optionally, the capturing portion has a thread, further optionally an external thread. The translating portion has a corresponding thread, further optionally an internal thread. As such, based on the corresponding threads of the capturing portion and of the translating portion, a screwed connection between the capturing portion and the translating portion can be established. As such, the translating portion, for example, a telescopic tube thereof, is fixed to the capturing portion, restricting any movement relative to each other, be it rotation or translation.

Optionally, the capturing portion is provided at the rear end of the holder. The rear end of the holder is the end of the holder facing towards the operator and away from the patient.

Optionally, the holder, in particular the connecting portion thereof, is configured to connect at its front end to the rear end of the handle. As such, a compact configuration of the handle and of the holder can be achieved.

Optionally, the holder comprises the portions in the following order from the front end to the rear end of the holder, in particular in the shortened state as well as in the extended state, i.e. along the length direction: connecting portion, translating portion, capturing portion.

The disclosure is also directed to a set comprising the holder as described above and a handle, optionally comprising a catheter. More specifically, the connecting portion of the holder connects to the handle. The handle and the holder may be integrally formed, and may, in particular, be formed as one piece.

The connecting portion is located opposite an outlet for the catheter from the handle, optionally wherein the handle is configured such that the guidewire extends through the handle in the length direction. As such, the holder forms a backwards extension on handle, the the guidewire substantially extending along the length direction with no change of direction along the handle and the holder.

The holder and/or the handle are specifically configured for thrombectomy or atherectomy applications. Therefore, the present disclosure also concerns atherectomy or thrombectomy systems comprising the holder or the set as explained above.

### BRIEF DESCRIPTION OF THE DRAWINGS

FIG. 1 shows a cross-section of a holder of the present disclosure.

FIG. 2a shows the holder in the extended state.

FIG. 2b shows the holder in the shortened state.

FIG. 3a shows a first embodiment of the holder.

FIG. 3b shows a second embodiment of the holder.

### DETAILED DESCRIPTION

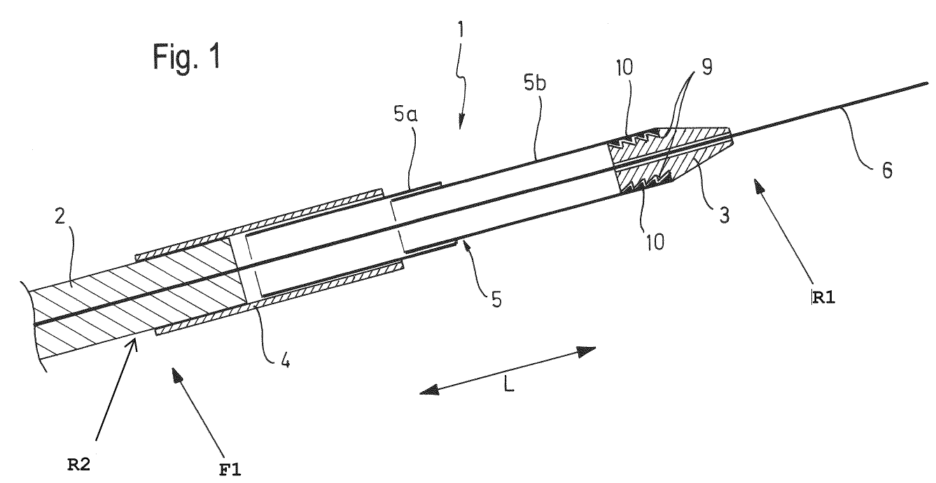

A holder 1 for holding and positioning a guidewire 6 is shown in FIG. 1. The guidewire 6 is held relative to a handle 2 by way of the holder 1 during a vascular treatment. The holder 1 has a capturing portion 3, a connecting portion 4 and a translating portion 5. The capturing portion 3 is connected to the translating portion 5, and the translating portion 5 is connected to the connecting portion 4. In particular, the translating portion 5 is, at least a part of it, fixed relative to the capturing portion 3, and another portion of the translating portion 5 is fixed relative to the connecting portion 4.

The capturing portion 3 captures the guidewire 6 so as to restrict movement, in particular rotation and translation, of the guidewire 6 relative to the capturing portion 3.

The connecting portion 4 connects the holder 1 to the handle 2 and restricts movement, in particular rotation, between the connecting portion 4 and the handle 2.

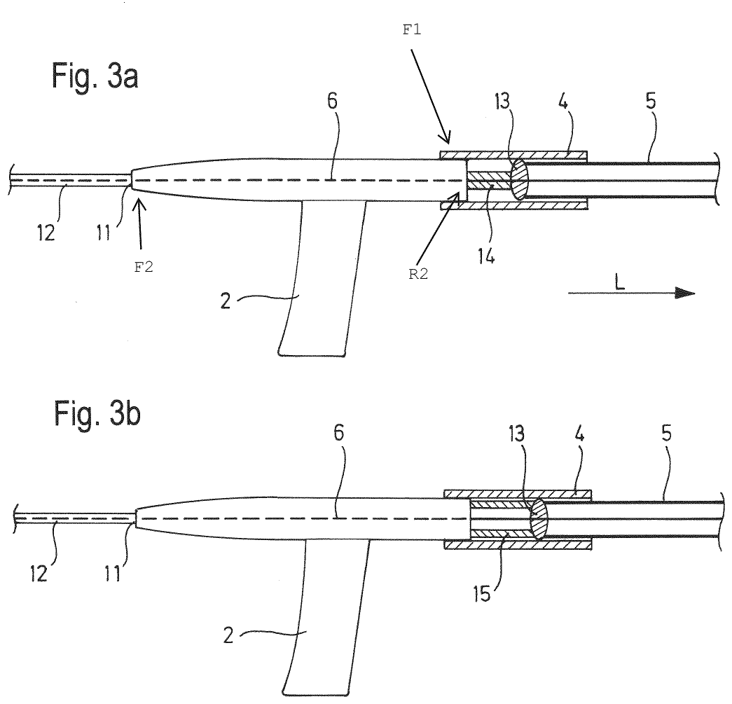

The translating portion 5 translates the capturing portion 3 relative to the connecting portion 4 between a shortened state and an extended state of the holder 1 along a length direction L, while e guidewire 6 is captured by the capturing portion 3. The translating portion 5 shortens and extends the holder 1 in the length direction in the shortened and extended states along the arrow L Accordingly, the holder 1 allows to facilitate retraction and advancement of a catheter 12 (FIGS. 3a and 3b) relative to the guidewire 6 in the length direction while restricting rotation of the guidewire 6 relative to the holder 1 and the handle 2.

When the handle 2 is advanced towards the patient, the catheter 12 (FIGS. 3a and 3b) is advanced relative to the guidewire 6 and particularly advanced relative to the capturing portion 3, which both remain stationary. By doing so, the holder 1 moves into the extended position, see FIG. 1.

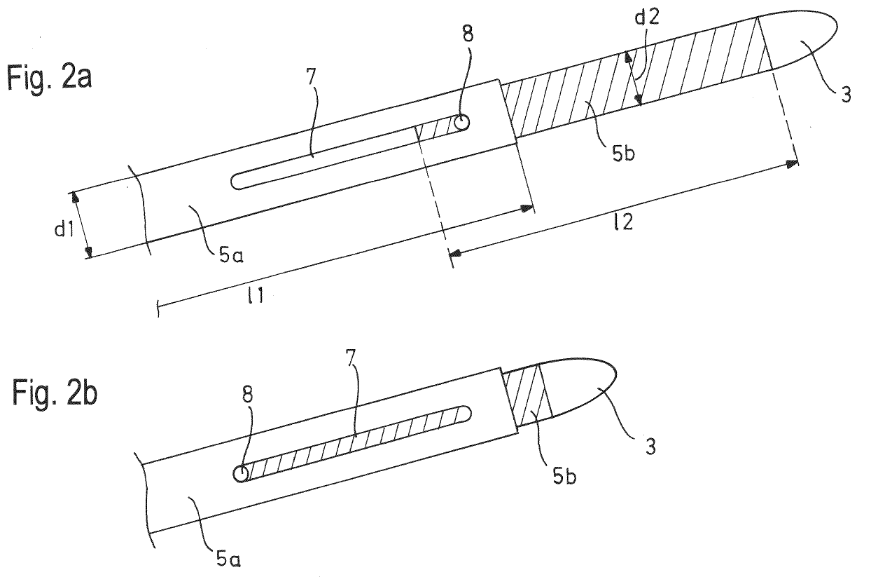

Vice-versa, when the handle 2 is retracted, i.e. moved away from the patient, the catheter 12 (FIGS. 3a and 3b) is retracted relative to the guidewire 6 and particularly the capturing portion 3. As such, the holder 1 is moved into the shortened position, as shown in FIG. 2b.

By doing so, i.e. by repeatedly advancing and retracting the handle, the relative positions between the catheter 12 and the guidewire 6 can be modified along the treatment area as needed during the procedure, i.e. back and forth movement of the catheter 12 can be carried out while the position of the guidewire 6 is kept stationary.

As can be seen in FIG. 1, the guidewire 6 extends through the holder 1 in the length direction L. The translating portion 5 houses a part of the guidewire 6.

The translating portion 5 includes two telescopic elements 5a and 5b. The telescopic elements are tubular and are tubes 5a and 5b, as shown in FIG. 1. Tube 5a telescopes relative to tube 5b in the length direction L, and vice versa. The capturing portion 3 is stationary relative to the tube 5b, and the connecting portion 4 is stationary relative to the tube 5a. Generally, the telescopic tubes 5a and 5b may have a length between 50 mm and 300 mm and/or may have the same length.

The telescopic tubes 5a and 5b have outer diameters d1 and d2, respectively, of less than 20 mm, in particular less than 15 mm. The diameters are indicated in FIG. 2a.

More specifically, the outer tube 5a may have an inner diameter between 8 mm and 13 mm, and/or the inner telescopic tube 5b may have an outer diameter between 5 mm and 10 mm. As regards the cross-sectional areas of the tubes (or the telescopic elements in general), an inner cross-sectional area of the outer telescopic element may be between 50 and 140 mm² while an outer cross-sectional area of the inner telescopic element may be between 20 and 80 mm².

The telescopic tube 5b inner telescopic element, while the telescopic tube 5a represents an outer telescopic element that receives the inner telescopic element 5b at least partially. The alternative arrangement with respect to inner and outer positions is also conceivable, meaning that the capturing portion 3 may be stationary relative to an outer tube, and the connecting portion 4 may be stationary relative to an inner tube.

With reference to FIG. 1, the connecting portion 4 establishes a friction fit relative to the handle 2. Hence, by way of the connecting portion 4, the telescopic tube 5a is fixed relative to the handle 2, and the entire holder 1 may be regarded as connected to the handle 2 via the connecting portion 4.

The capturing portion 3 establishes a friction fit relative to the guidewire 6. Accordingly, at the capturing portion 3, the guidewire 6 is fixed relative to the telescopic tube 5b, and, as such, connected to the holder 1.

The capturing portion 3 establishes a form fit relative to the translating portion 5. More specifically, the capturing portion 3 establishes a form fit relative to the telescopic element 5b. The capturing portion 3 has an external thread 9, while the tube 5b has an internal thread. By way of the threads, a screwed connection between the capturing portion 3 and the translating portion 5, more specifically, the telescopic element 5b, can be established. As such, the telescopic elements 5a and 5b can move relative to each other in the length direction L. Therefore, the capturing portion 3 and the telescopic element 5a can move relative to each other in the length direction L.

As can be taken from FIG. 1, the capturing portion 3 is provided at the end R1 of the holder 1. This end can be regarded as the rear end R1 of the holder 1.

As shown in FIG. 1, the holder 1 comprises the connecting portion 4, the translating portion 5 and the capturing portion 3 in the direction from the left side to the right side, i.e. from a position nearest a patient to a position furthest a patient in the length direction L. With respect to the handle 2, the holder 1, in particular the connection portion 4 thereof, connects at its front end F1 to the rear part R2 of the handle 2.

FIGS. 2a and 2b show the holder 1 in the extended and shortened states, respectively. As can be taken from FIGS. 2a and 2b, the first telescopic element 5a comprises, at least partially along its length, a receiving feature 7. The receiving feature 7 may be a channel extending in the length direction L of the telescopic element 5a. The second telescopic element 5b comprises an engagement feature 8, which may be a protrusion. The engagement feature 8 is at least partially received within the receiving feature 7, such that rotation of the telescopic elements 5a and 5b relative to each other is restricted and (only) translation of the telescopic elements 5a and 5b relative to each other in the length direction L is possible. The receiving feature 7 and the engagement feature 8 correspond to each other and allow for engagement while facilitating movement of the engagement feature 8 in the length direction L within and along the receiving feature 7. Accordingly, when the engagement feature 8 is in the extended position, as shown in FIG. 2a, the holder is in the extended state.

If the engagement feature 8 is in the shortened position, the first and second telescopic tubes 5a and 5b almost entirely overlap, so that the holder 1 is in the shortened state, as shown in FIG. 2b.

As the engagement feature 8 can only move in the length direction L relative to the receiving feature 7, and the engagement between the receiving feature 7 and the engagement feature 8 prohibits rotational movement relative to each other, it is secured that the telescopic elements 5a and 5b and, hence, the translating portion 5, allow(s) for translational movement, i.e. movement in the length direction L only. This avoids undesired rotational movement of the guidewire 6 relative to the handle 2.

With continued reference to FIG. 2a, the telescopic element 5a has a length l1, while the length of the telescopic element 5b is l2. Outer diameters d1 and d2 of tubes 5a and 5b, respectively, are indicated in FIG. 2a as well.

The connecting portion 4, the capturing portion 3 and/or the translating portion 5 can be separate pieces. Alternatively, these portions can be a single piece, and can be integrally formed. Furthermore, the holder 1 can be integrally formed with the handle 2.

The disclosure also confers to a kit or set comprising the holder 1 and the handle 2.

FIGS. 3a and 3b show a set comprising the handle 2 (comprising the catheter 12) and the holder 1 (FIG. 1). By way of the connecting portion 4, the handle 2 and the holder 1 are connected. In an alternative configuration (not shown in the drawings) the handle 2 and the holder 1 may be integrally formed.

The handle 2 has an outlet l1, where the catheter 12 is provided and extends in the distal direction, i.e. towards the patient, which is the left-direction in FIGS. 3a and 3b. The connecting portion 4 is connected to the translating portion 5, more specifically to the tube 5a (FIG. 1). The handle 2 comprises a guidewire adaptor 13 which acts inside the handle 2 on the guidewire 6 by capturing and fixing the guidewire 6. By way of a clip 14, as shown in FIG. 3a, the guidewire adaptor 13 remains in an extended position, as needed during the procedure, i.e. during use of the catheter 12.

Alternatively, for positioning and keeping the guidewire adaptor 13 in the extended position, the translating portion 5, as shown in FIG. 3b, may be formed so that no clip 14 is needed any more. For example, the translating portion 5 can have an adaptor feature 15 at its front part, the adaptor feature 15 decreasing the diameter of the space within the connecting portion 4 forward of the translating portion 5. In other words, the free space inside the connecting portion 4 is reduced, so that the guidewire adaptor 13 is stopped from being retracted towards the handle 2. Accordingly, instead of the clip 14, the adaptor feature 15 at the translating portion 5 ensures that the desired position of the guidewire adaptor 13 is maintained.

With continued reference to FIGS. 3a and 3b, the handle 2 comprises an outlet l1 for a catheter 12. The connecting portion 4 of the holder 1 (FIG. 1) is located opposite the outlet 11 at the handle 2. The handle 2 is configured such that the guidewire 6 extends through the handle 2 and the holder 1 in the length direction L. Hence, ideally, the guidewire 6 does not need to change the direction, but extends straight along the length direction L from the outlet 11, where the guidewire 6 enters the handle 2 and extends inside the holder 1.

The holder 1 of the present disclosure or the set of the holder 1 and the handle 2 (optionally having the catheter 12) of the present disclosure is part of an atherectomy or thrombectomy system (not shown in the drawings). The atherectomy or thrombectomy system comprises, in addition to the holder 1 and the handle 2, a control unit, optionally the catheter 12, an aspiration feature allowing for aspiration of clot and calcifications from the occlusion at the site, and a rotating feature (helix) within the catheter 12 (none of these entities shown in the drawings). By way of rotation, aspiration and maceration of the clot or calcification as removed from the site is improved. For example, a rotating helix extending along the entire length of the catheter can be used. In particular, for atherectomy applications, also the most distal end of the catheter 12 can rotate. Specifically, a rotating head can be provided, which helps to assist in removing material from the vasculature. The holder 1 of the present disclosure is in particular useful for such applications, as rotation of the guidewire 6 is restricted at the same time.

### LIST OF REFERENCE SIGNS

1 Holder
2 Handle
3 Capturing portion
4 Connecting portion
5 Translating portion
5a, 5b Telescopic elements
6 Guidewire
7 Receiving feature
8 Engagement feature
9 Thread of capturing portion
10 Thread of translating portion
11 Outlet
12 Catheter
13 Guidewire adaptor
14 Clip
15 Adaptor feature
L length direction
F1 front end of holder
R1 rear end of holder
F2 front end of handle
R2 rear end of handle
d1, d2 outer diameters
l1, l2 length

1. A holder for connecting to a handle and for holding a guidewire and positioning a catheter relative to the guidewire during a vascular treatment, the holder comprising a capturing portion, a connecting portion, and a translating portion, wherein the capturing portion is connected to the translating portion, and the translating portion is connected to the connecting portion,
the capturing portion configured for capturing the guidewire to restrict movement, in particular rotation and translation, of the guidewire relative to the capturing portion,
the connecting portion configured for connecting the holder to the handle to restrict movement, in particular rotation, between the connecting portion and the handle,
the translating portion configured for translating the capturing portion relative to the connecting portion between a shortened state and an extended state of the holder along a length direction, while the guidewire is captured by the capturing portion, wherein the translating portion is configured to shorten and extend the holder in the length direction, while rotation of the translating portion relative to the capturing portion and the connecting portion is restricted,
such that the holder is configured to facilitate relative movement between the catheter and the guidewire in the length direction while restricting rotation of the guidewire relative to the handle.

2. The holder of claim 1, wherein the translating portion is configured to house a section of the guidewire when the translating portion is in the retracted state or the extended state.

3. The holder of claim 1, wherein the translating portion includes at least two telescopic elements which are configured to telescope relative to each other in the length direction of the guidewire, the capturing portion being stationary relative to at least one of the at least two telescopic elements and the connecting portion being stationary relative to the other one of the at least two telescopic elements.

4. The holder of claim 3, wherein each of the telescopic elements has a length between 50 and 300 mm.

5. The holder of claim 3, wherein each of the telescopic elements are tubular and each have a diameter of less than 20 mm.

6. The holder of claim 3, wherein the at least two telescopic elements comprise an inner telescopic element and an outer telescopic element at least partially receiving the inner telescopic element.

7. The holder of claim 3, wherein one of the at least two telescopic elements at least partially comprises a receiving feature extending along a length direction of the telescopic elements; and the other one of the at least two telescopic elements comprises an engagement feature at least partially received in the receiving feature such that rotation of the telescopic elements relative to each other is restricted and translation of the telescopic elements relative each other in the length direction is possible.

8. The holder of claim 1, wherein the connecting portion is configured to establish a friction fit relative to the handle.

9. The holder of claim 1, wherein the capturing portion is configured to establish a friction fit relative to the guidewire.

10. The holder of claim 1, wherein the capturing portion is configured to establish a form fit relative to the translating portion.

11. The holder of claim 1, wherein the capturing portion is provided at a rear end of the holder.

12. The holder of claim 1, wherein the holder, in particular the connecting portion, is configured to connect at its front end to a rear end of the handle.

13. The holder of of claim 1, wherein components are positioned in the following order from a front end to a rear end of the holder: connecting portion, translating portion, capturing portion.

14. A set comprising the handle and the holder of claim 1, wherein the connecting portion (4) connects the handle and the holder.

15. The set of claim 14, wherein the connecting portion is located opposite an outlet for the catheter from the handle.

16. An atherectomy or thrombectomy system comprising the holder of claim 1.
